(19) United States
(12) Patent Application Publication
(10) Pub. No.: US 2024/0079116 A1
(43) Pub. Date: Mar. 7, 2024

(54) AUTOMATED SEGMENTATION OF ARTIFACTS IN HISTOPATHOLOGY IMAGES

(71) Applicant: Ventana Medical Systems, Inc., Tuscon, AZ (US)

(72) Inventors: Mohammad Saleh MIRI, San Jose, CA (US); Aicha BEN TAIEB, Mountain View, CA (US); Uday Kurkure, Sunnyvale, CA (US)

(73) Assignee: Ventana Medical Systems, Inc., Tuscon, AZ (US)

(21) Appl. No.: 18/499,208

(22) Filed: Oct. 31, 2023

Related U.S. Application Data
(63) Continuation of application No. PCT/US2022/030395, filed on May 20, 2022.
(60) Provisional application No. 63/191,567, filed on May 21, 2021.

(51) Int. Cl. G16H 30/40; G06T 7/12; G06T 7/174

(52) U.S. Cl. CPC G16H 30/40; G06T 7/12; G06T 7/174; G06T 2207/20084

(57) ABSTRACT

Techniques for image segmentation of a digital pathology image may include accessing an input image that depicts a section of a tissue; and generating a segmentation image by processing the input image using a generator network, the generator network having been trained using a data set that includes a plurality of pairs of images. The segmentation image indicates, for each of a plurality of artifact regions of the input image, a boundary of the artifact region. At least one of the plurality of artifact regions depicts an anomaly that is not a structure of the tissue. Each pair of images of the plurality of pairs includes a first image of a section of a tissue, the first image including at least one artifact region, and a second image that indicates, for each of the at least one artifact region of the first image, a boundary of the artifact region.

### CROSS-REFERENCE TO RELATED APPLICATIONS

This application is a continuation of International Application No. PCT/US2022/030395 filed on May 20, 2022, which claims the benefit of and priority to U.S. Provisional Patent Application No. 63/191,567 filed on May 21, 2021, each of which are hereby incorporated by reference in their entireties for all purposes.

### FIELD

The present disclosure relates to digital pathology, and in particular to techniques that include semantic segmentation of a digital pathology image.

### BACKGROUND

Histopathology may include examination of slides prepared from sections of tissue for a variety of reasons, such as: diagnosis of disease, assessment of a response to therapy, and/or the development of pharmacological agents to fight disease. Because the tissue sections and the cells within them are virtually transparent, preparation of the slides typically includes staining the tissue sections in order to render relevant structures more visible. Digital pathology may include scanning of the stained slides to obtain digital images, which may be subsequently examined by digital pathology image analysis and/or interpreted by a human pathologist.

In addition to one or more regions to be analyzed, a digital pathology slide may include regions to be excluded from further analysis. Such regions may include, for example, regions that may be distracting during the task of annotating a tumor region and/or regions that may produce spurious results if not excluded from an automated scoring operation. The task of manually annotating a slide to indicate regions to be excluded is expensive, time-consuming, and subjective.

### SUMMARY

In various embodiments, a computer-implemented method of image segmentation is provided that includes accessing an input image that depicts a section of a tissue and includes a plurality of artifact regions; and generating a segmentation image by processing the input image using a generator network, the generator network having been trained using a training data set that includes a plurality of pairs of images. The segmentation image indicates, for each of the plurality of artifact regions of the input image, a boundary of the artifact region. In this method, at least one of the plurality of artifact regions depicts an anomaly that is not a structure of the tissue, and, for each pair of images of the plurality of pairs of images, the pair includes a first image of a section of a tissue, the first image including at least one artifact region, and a second image that indicates, for each of the at least one artifact region of the first image, a boundary of the artifact region.

In some embodiments, the anomaly is a focus blur, a fold in the section of the tissue, a deposit of pigment in the section of the tissue, or matter trapped between the section of the tissue and a slide cover.

In some embodiments, the segmentation image comprises a binary segmentation mask.

In some embodiments, the method further comprises producing an annotated image that includes the segmentation image overlaid on the input image.

In some embodiments, the method further comprises estimating a quality of the input image, based on a total area of the plurality of artifact regions.

In some embodiments, the input image includes a second plurality of artifact regions, and the method further comprises generating a second segmentation image by processing the input image using a second generator network, the second generator network having been trained using a second training data set that includes a second plurality of pairs of images. The second segmentation image indicates, for each of the second plurality of artifact regions of the input image, a boundary of the artifact region, and at least one of the second plurality of artifact regions depicts a biological structure of the tissue.

In some embodiments, the computer-implemented method further comprises determining, by a user, a diagnosis of a subject based on the segmentation image.

In some embodiments, the computer-implemented method further comprises administering, by the user, a treatment with a compound based on (i) the segmentation image, and/or (ii) the diagnosis of the subject.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Aspects and features of the various embodiments will be more apparent by describing examples with reference to the accompanying drawings, in which:

FIG. 1 shows an example diagram of a digital pathology solution workflow;

FIG. 2A shows an example diagram of another digital pathology solution workflow;

FIG. 2B shows an example diagram of a digital pathology image;

FIG. 3 shows an example image of a thickness artifact;

FIG. 4 shows an example image of a pigment artifact;

FIG. 5 shows an example image of a tissue fold artifact;

FIG. 6 shows an example image of a dirt artifact;

FIG. 7 shows an example image of an air bubble artifact;

FIG. 8 shows an example image of a pen mark artifact;

FIG. 9 shows an example of a manually annotated digital pathology image;

FIG. 10 illustrates a flowchart for an exemplary process according to some embodiments;

FIG. 11 shows an example of automated segmentation of tissue folds according to some embodiments;

FIG. 12 shows an example of human-annotated segmentation of tissue folds;

FIG. 13A shows an example of an application according to some embodiments;

FIG. 13B shows an example of technician quality control of scanned tissue slides according to some embodiments;

FIG. 13C shows an example of automatic or visual tissue analysis according to some embodiments;

FIG. 14 shows an example of another application according to some embodiments;

FIG. 15A shows an example of a training workflow according to some embodiments;

FIG. 15B shows an example of whole slide annotation according to some embodiments;

FIG. 15C shows an example of training patches according to some embodiments;

FIG. 15D shows an example of a neural network architecture according to some embodiments;

FIG. 16 shows an example computing environment for segmenting an input image according to various embodiments;

FIGS. 17, 18, and 19 show an example of automated segmentation of tissue folds and focus blur regions according to some embodiments;

FIGS. 20, 21, and 22 show another example of automated segmentation of tissue folds and focus blur regions according to some embodiments; and

FIGS. 23, 24, and 25 show a further example of automated segmentation of tissue folds and focus blur regions according to some embodiments;

FIG. 26 shows example images of focus blur artifacts; and

FIG. 27 shows example images of stitching artifacts.

### DETAILED DESCRIPTION

Systems, methods and software disclosed herein facilitate segmentation of artifact regions within digital pathology images (e.g., WSIs). While certain embodiments are described, these embodiments are presented by way of example only, and are not intended to limit the scope of protection. The apparatuses, methods, and systems described herein may be embodied in a variety of other forms. Furthermore, various omissions, substitutions, and changes in the form of the example methods and systems described herein may be made without departing from the scope of protection.

I. Overview

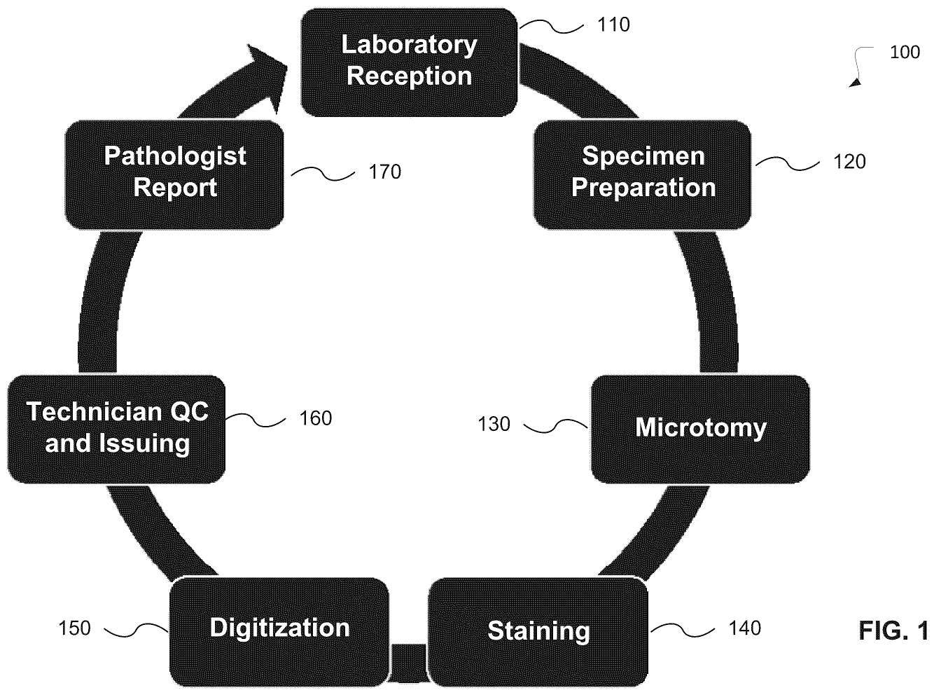

Digital pathology may involve the interpretation of digitized images in order to correctly diagnose subjects and guide therapeutic decision making. In digital pathology solutions, image-analysis workflows can be established to automatically detect or classify biological objects of interest (e.g., positive/negative tumor cells, etc.). FIG. 1 shows an example diagram of a digital pathology solution workflow 100. The digital pathology solution workflow 100 includes receiving a specimen at the laboratory at block 110, preparing the specimen (e.g., fixation, dehydration, clearing, wax infiltration, embedding) at block 120, microtomy (e.g., slicing the prepared specimen to obtain one or more tissue sections) at block 130, staining the tissue section(s) at block 140, digitizing (e.g., scanning) the prepared slides at block 150, technician quality control (QC) and issuing of the slide images (e.g., WSIs) at block 160, and reporting of analysis results (e.g., diagnosis) by a pathologist at block 240.

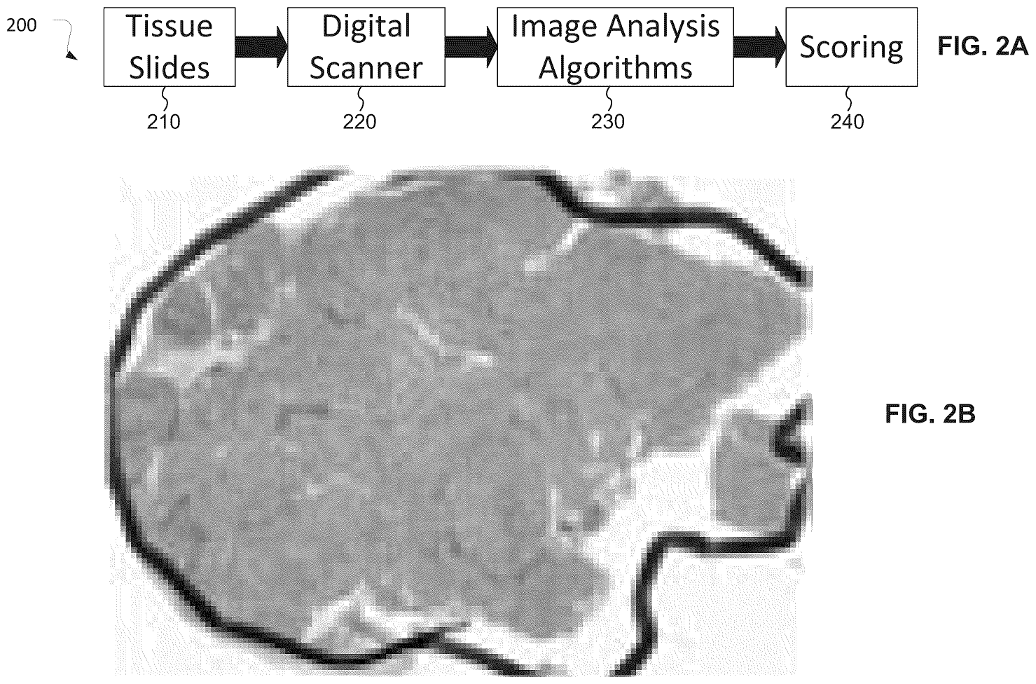

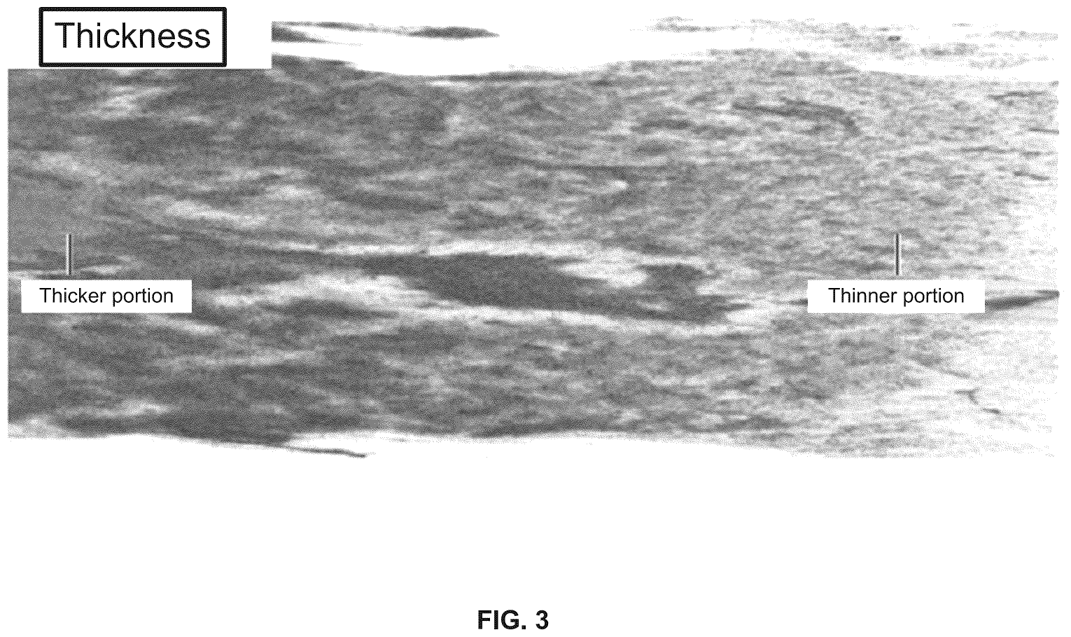

FIG. 2A shows another example diagram of a digital pathology solution workflow 200. The digital pathology solution workflow 200 includes obtaining tissue slides at block 210, scanning preselected areas or the entirety of the tissue slides with a digital image scanner (e.g., a whole slide image (WSI) scanner) to obtain digital images at block 220, performing image analysis on the digital image using one or more image analysis algorithms at block 230, and scoring objects of interest based on the image analysis (e.g., quantitative or semi-quantitative scoring such as positive, negative, medium, weak, etc.) at block 240.

Evaluation of tissue changes caused, for example, by disease, may be performed by examining thin tissue sections. A tissue sample (e.g., a sample of a tumor) may be sliced to obtain a series of sections, with each section having a thickness of, for example, 4-5 microns. Because the tissue sections and the cells within them are virtually transparent, preparation of the slides typically includes staining the tissue sections in order to render relevant structures more visible. For example, different sections of the tissue may be stained with one or more different stains to express different characteristics of the tissue.

Each section may be mounted on a slide, which is then scanned to create a digital image that may be subsequently examined by digital pathology image analysis and/or interpreted by a human pathologist (e.g., using image viewer software). The pathologist may review and manually annotate the digital image of the slides (e.g., tumor area, necrosis, etc.) to enable the use of image analysis algorithms to extract meaningful quantitative measures (e.g., to detect and classify biological objects of interest). Conventionally, the pathologist may manually annotate each successive image of multiple tissue sections from a tissue sample to identify the same aspects on each successive tissue section. FIG. 2B shows an example of a digital pathology image that has been annotated by drawing a boundary in pen.

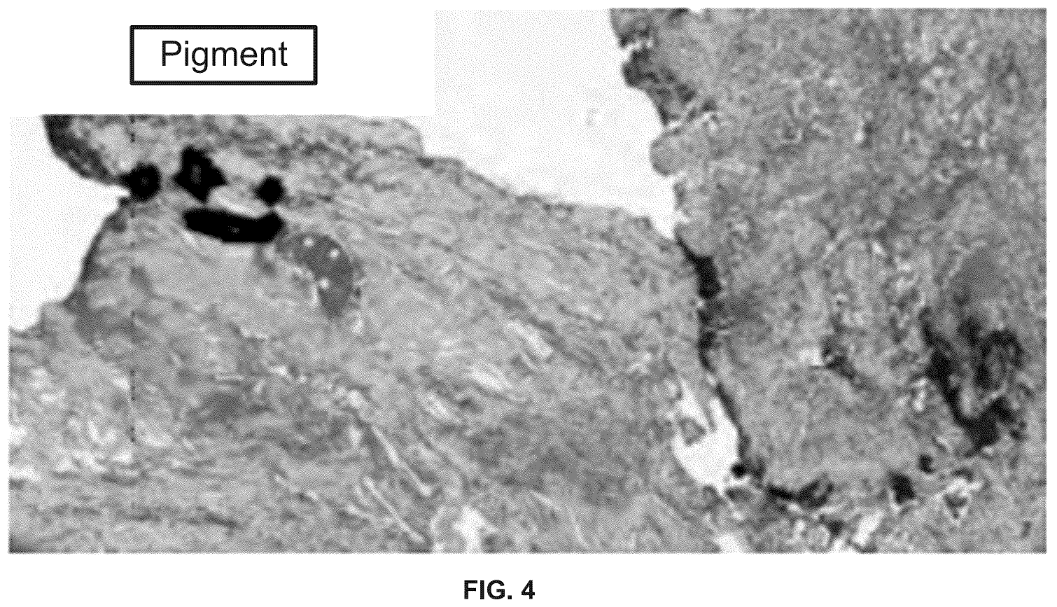

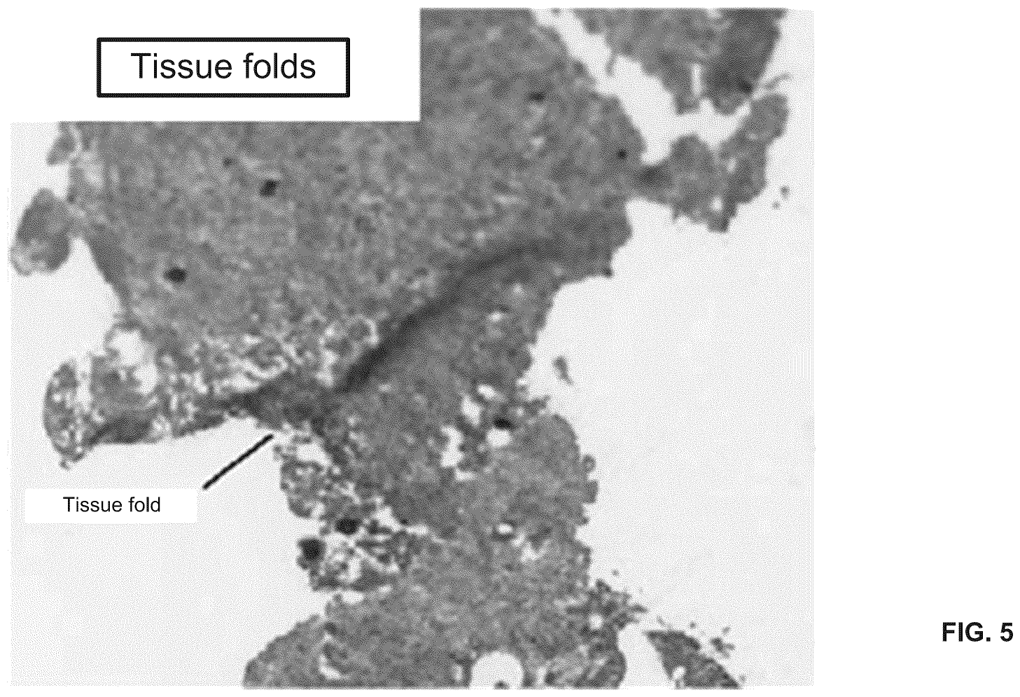

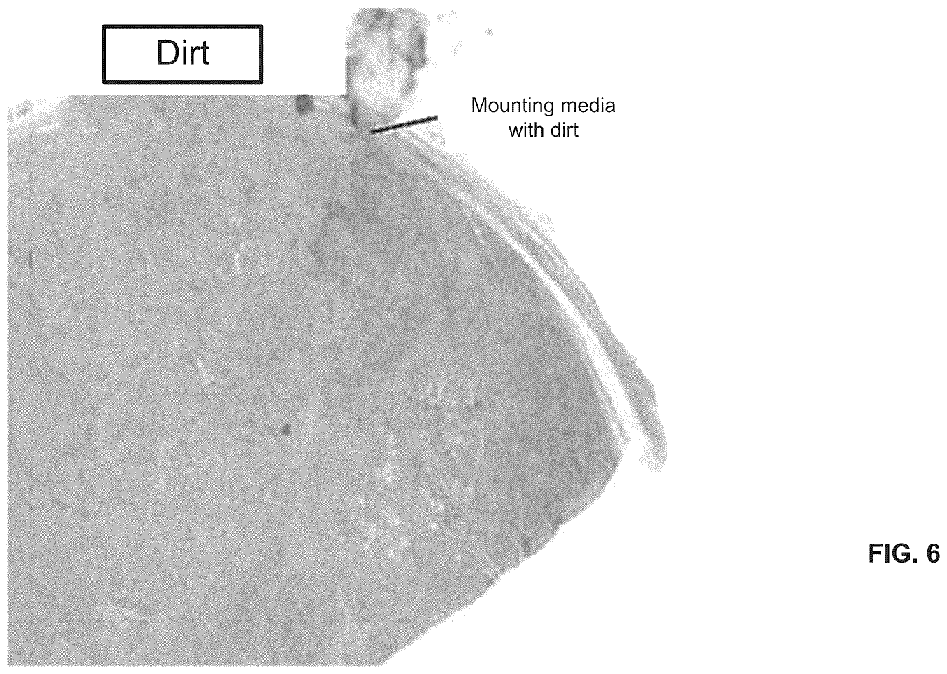

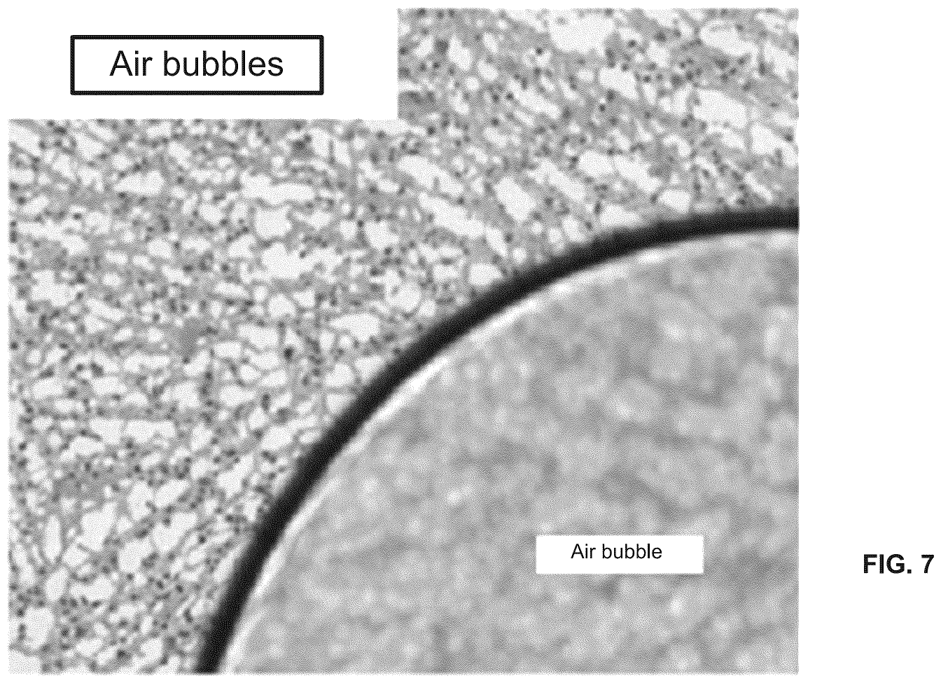

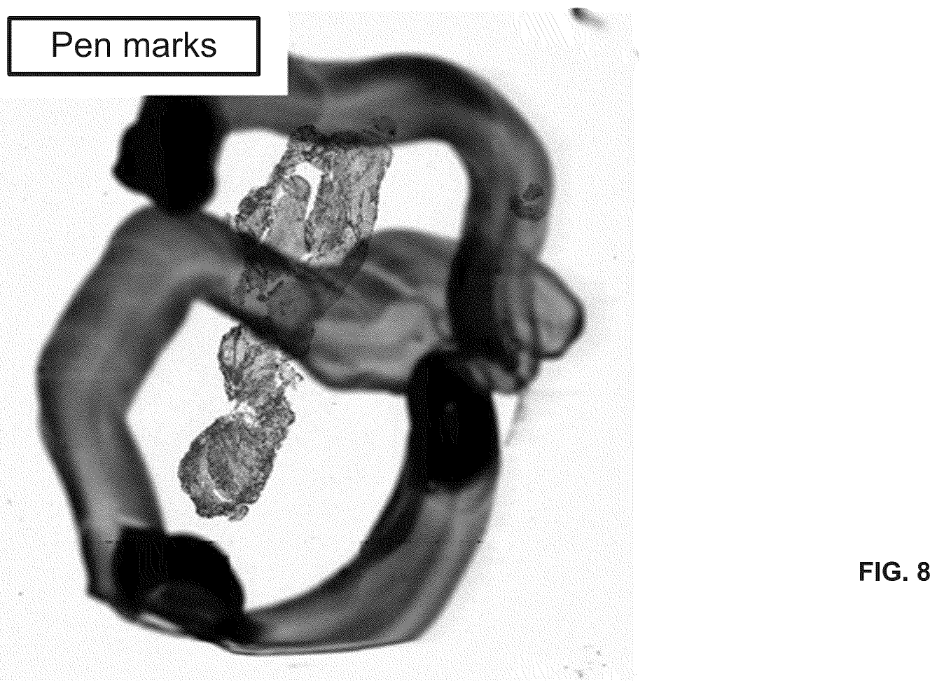

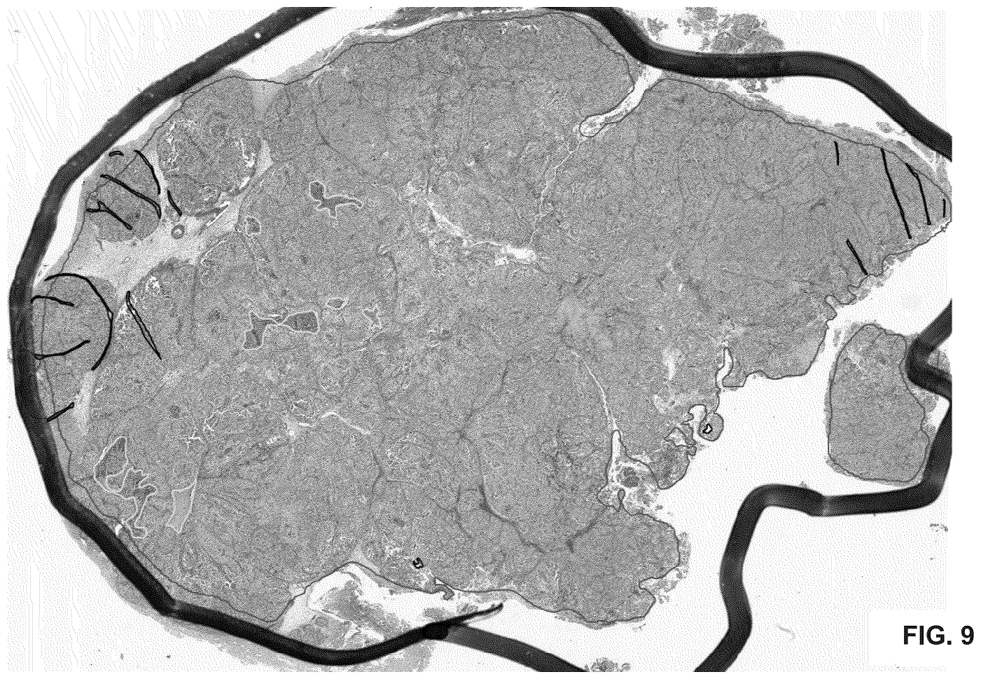

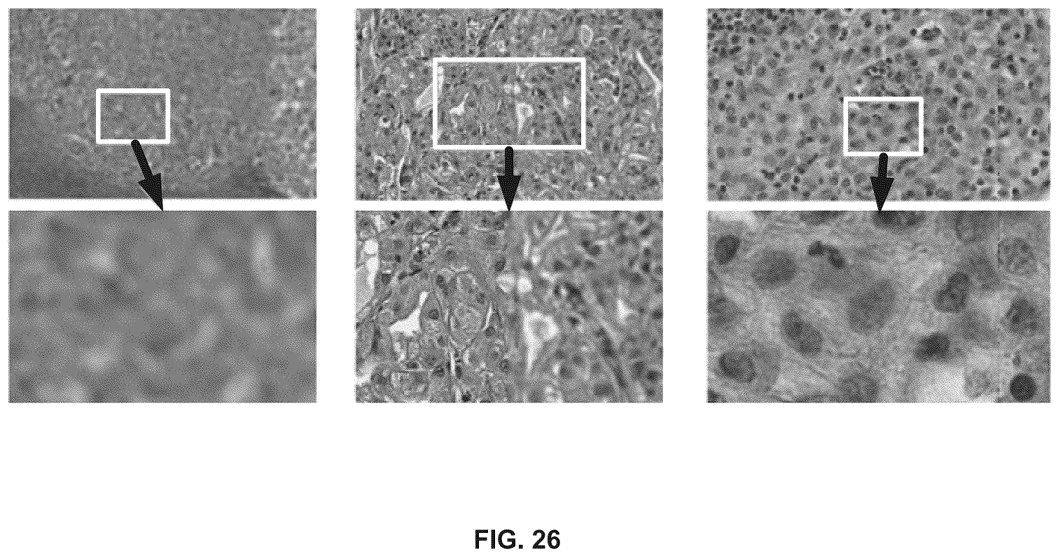

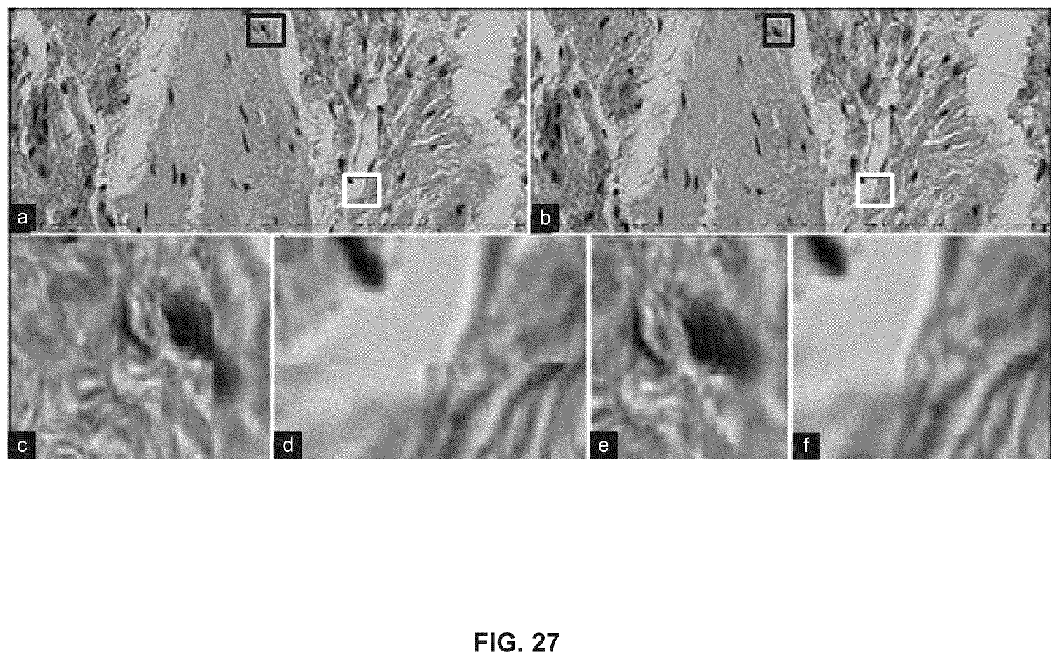

A stained section of a tissue sample on a histological slide may have various types of defects that may obscure the information to be conveyed. Such defects may be due to causes which may arise during tissue preparation: for example, the tissue section may be thicker in one part than in another (FIG. 3 shows one example), and/or the section may include one or more pigment deposits (e.g., precipitates) (FIG. 4 shows one example). Other defects may be due to causes that may arise during slide preparation: for example, the tissue section may be folded on the slide (FIG. 5 shows one example), and/or unwanted matter (e.g., one or more foreign particles, such as dirt or other debris; one or more air bubbles) may be trapped between the tissue section and the slide cover (FIGS. 6 and 7 show examples). Other defects may be due to causes that may arise during human handling and/or automated processing (e.g., scanning) of a prepared slide: for example, pen marks (FIG. 8 shows one example), focus blur (i.e., out-of-focus) (FIG. 26 shows three examples), and/or false features created during digital stitching of individual scan tiles to obtain the WSI (FIG. 27 shows several examples, in which the black and white boxes in an image are enlarged in detail images c and d, respectively, and the black and white boxes in image b are enlarged in detail images e and f, respectively). Such defects are examples of “artifacts,” in that a part of the image depicts something (e.g., a structure) that was not actually present in the tissue. To support accurate analysis of the slide images, it may be desirable to detect such artifacts and, if possible, to process the image to improve the extent to which it conveys accurate information about a subject, such that interpretations of such information (e.g., for pathological diagnosis, prognosis, and/or treatment selection) can be improved.

To this end, current practice may include evaluation of digital-pathology images by a human pathologist to assess quality (e.g., of the image, of the section, and/or in general) before the images of the stained sample sections are analyzed (e.g., to detect and/or characterize particular biological objects or particular biomarkers in the stained sample sections). If the quality of the stained sample sections is poor, the corresponding digital pathology image may be discarded from a digital-pathology analysis performed for a given subject. However, detecting artifacts in digital pathology images can be both subjective and time-consuming.

Image artifacts as noted above (also called “anomalies”) are an important challenge in the adoption of a digital pathology (DP) workflow (e.g., as shown in FIGS. 1 and/or 2A). The artifacts may lead to a low quality of images of the stained sample sections, which may cause a misdiagnosis or delay in diagnosis. For example, multiple artifacts (e.g., out of focus, water marks, and tissue folds) present in an image of a stained sample may potentially obscure the diagnostic features. These artifacts may even lead to complete uselessness of the tissue. To accurately analyze the slide images, it may be desirable to detect such artifacts in the slide images, and, if possible, to process the slide images such that the artifacts do not interfere with the pathological diagnosis.

In addition to artifacts that may reduce the overall quality of a scanned image, a slide image may include regions that depict biological features (e.g., structures) of the tissue section and are to be excluded from subsequent analytical tasks. Examples of such features (also called “biological artifacts”) which are generally excluded from analysis include necrotic tissue, blood pools, and serum. Other such features may be excluded from subsequent analytical tasks on an application-specific basis. For example, it may be desired to exclude regions that depict macrophages in order to facilitate programmed death-ligand 1 (PD-L1) scoring of a slide that has been stained using an SP142 assay.

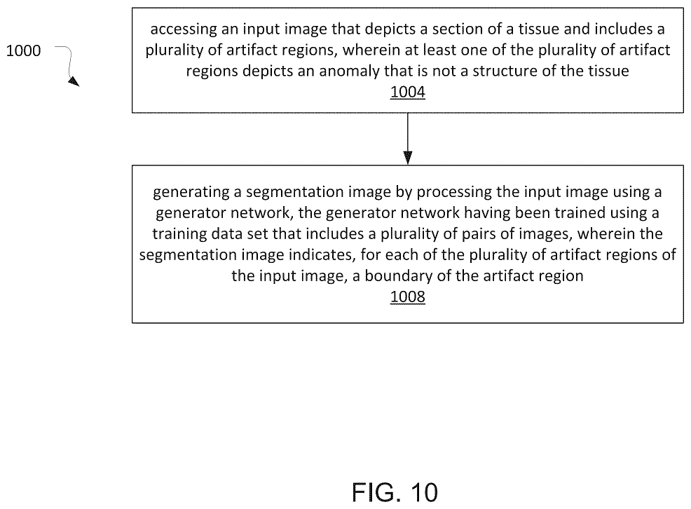

In current practice, the DP workflow relies on pathologists to visually inspect the digital slide image to identify artifacts, and to delineate such regions for exclusion from later analysis by manually annotating the image, which is a laborious and costly process. FIG. 9 shows an example in which the slide image of FIG. 2B has been manually annotated to delineate tissue folds (black), necrotic regions (yellow), and blood (cyan) for exclusion from the region of analysis (tumor, indicated by green).

Generation of the segmentation image may be performed by a trained generator network, which may include parameters learned while training a fully convolutional network (FCN). The FCN may further include an encoder-decoder network and may be configured as a U-Net.

One illustrative embodiment of the present disclosure is directed to a computer-implemented method of image segmentation that includes accessing an input image that depicts a section of a tissue and includes a plurality of artifact regions; and generating a segmentation image by processing the input image using a generator network, the generator network having been trained using a training data set that includes a plurality of pairs of images. The segmentation image indicates, for each of the plurality of artifact regions of the input image, a boundary of the artifact region. In this method, at least one of the plurality of artifact regions depicts an anomaly that is not a structure of the tissue, and, for each pair of images of the plurality of pairs of images, the pair includes a first image of a section of a tissue, the first image including at least one artifact region, and a second image that indicates, for each of the at least one artifact region of the first image, a boundary of the artifact region.

Advantageously, a method of image segmentation as described herein may be applied to optimize a digital pathology workflow at one or more different levels. In one example, such a method may be applied to provide a more scalable, robust and accessible image quality control (QC) algorithm. In another example, such a method may be applied to optimize pathologist annotation and review time. In a further example, such a method may be applied to optimize the performance of other downstream tasks (i.e., automatic image analysis).

II. Definitions

As used herein, when an action is “based on” something, this means the action is based at least in part on at least a part of the something.

As used herein, the terms “substantially,” “approximately” and “about” are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1, 1, 5, and 10 percent.

As used herein, the term “sample” “biological sample” or “tissue sample” refers to any sample including a biomolecule (such as a protein, a peptide, a nucleic acid, a lipid, a carbohydrate, or a combination thereof) that is obtained from any organism including viruses. Other examples of organisms include mammals (such as humans; veterinary animals like cats, dogs, horses, cattle, and swine; and laboratory animals like mice, rats and primates), insects, annelids, arachnids, marsupials, reptiles, amphibians, bacteria, and fungi. Biological samples include tissue samples (such as tissue sections and needle biopsies of tissue), cell samples (such as cytological smears such as Pap smears or blood smears or samples of cells obtained by microdissection), or cell fractions, fragments or organelles (such as obtained by lysing cells and separating their components by centrifugation or otherwise). Other examples of biological samples include blood, serum, urine, semen, fecal matter, cerebrospinal fluid, interstitial fluid, mucous, tears, sweat, pus, biopsied tissue (for example, obtained by a surgical biopsy or a needle biopsy), nipple aspirates, cerumen, milk, vaginal fluid, saliva, swabs (such as buccal swabs), or any material containing biomolecules that is derived from a first biological sample. In certain embodiments, the term “biological sample” as used herein refers to a sample (such as a homogenized or liquefied sample) prepared from a tumor or a portion thereof obtained from a subject.

III. Techniques for Automated Segmentation of Artifacts in a Digital Pathology Image

Applications for an automated approach for segmenting artifacts in digitized slides as described herein may include a tool to facilitate a pathology review and/or pathology scoring by removing regions to be excluded from a further analysis (e.g., an immunohistochemistry (IHC) estimation scoring workflow, a hematoxylin-and-eosin (H & E) tumor segmentation, a tumor estimation workflow). Such a tool may be implemented to output segmentation masks, apply the masks back onto the corresponding input images, and then input the masked images into an image analysis algorithm for further processing (e.g., to segment the tumor cells, count cells, etc.). An automated approach for segmenting artifacts in digitized slides as described herein may be implemented to be scanner-agnostic, tissue-agnostic, and/or stain-agnostic.

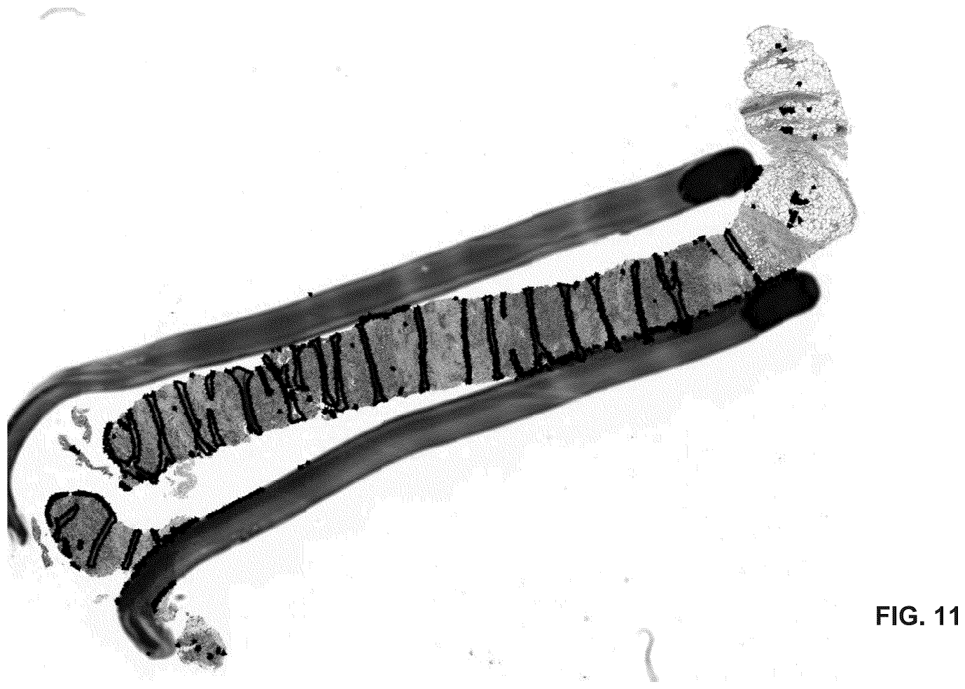

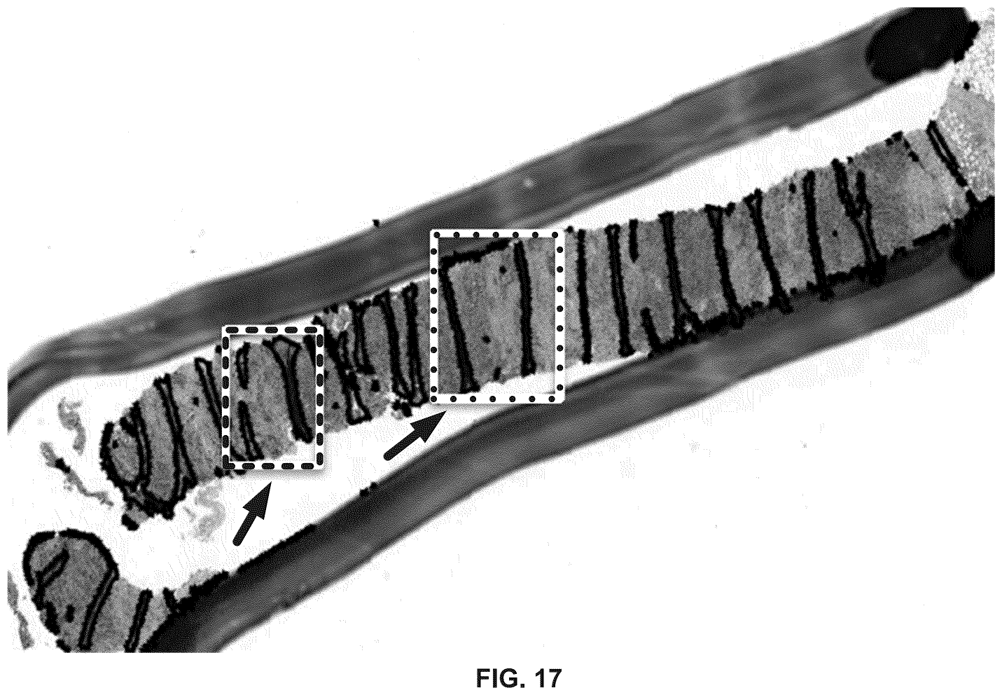

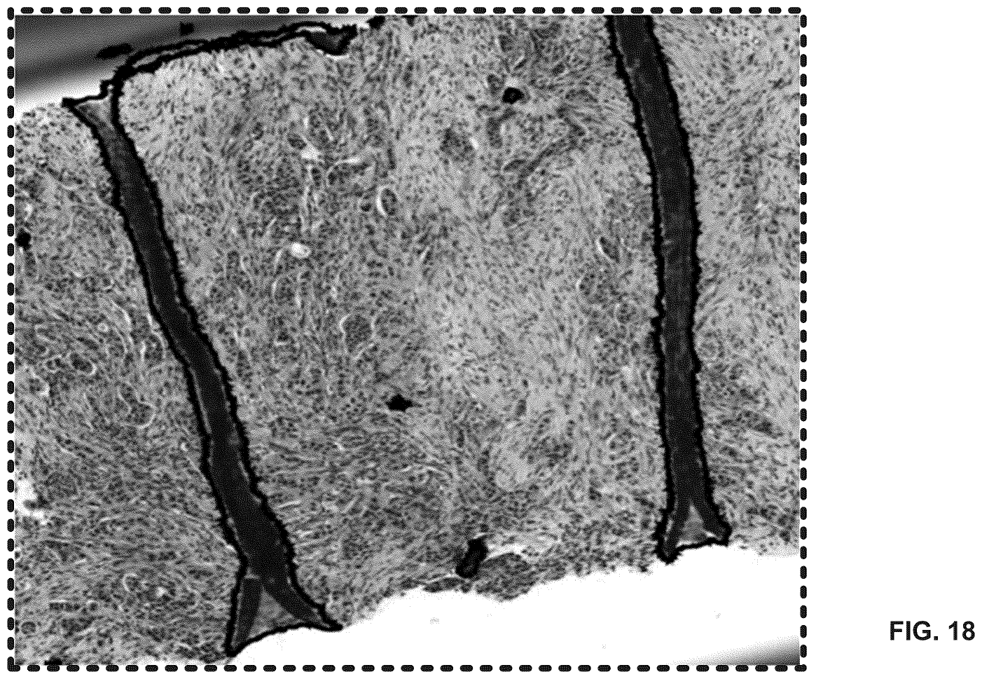

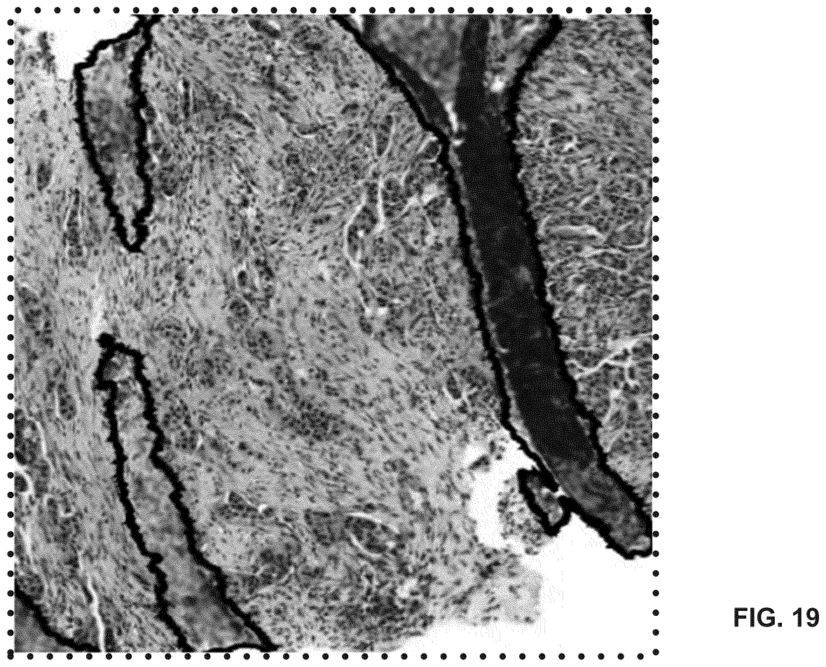

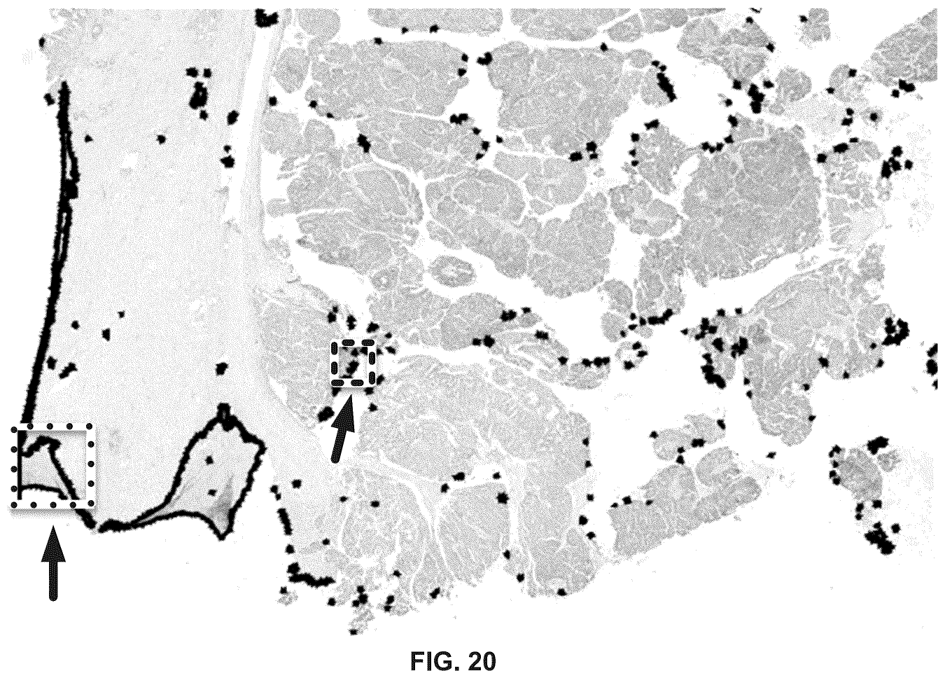

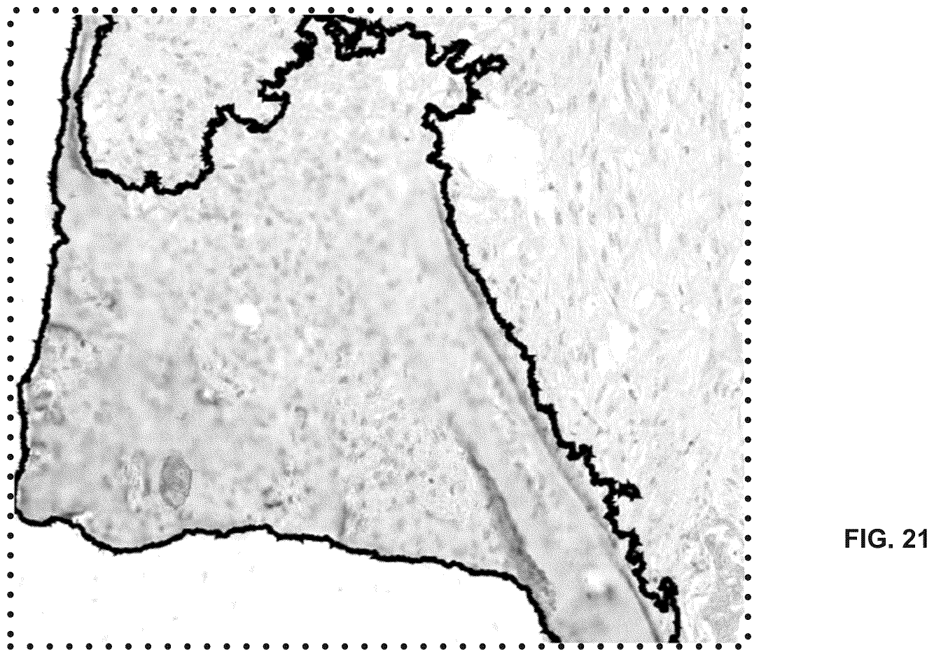

FIG. 10 illustrates a flowchart for an exemplary process 1000 for image segmentation according to some embodiments. Process 1000 may be performed using one or more computing systems, models, and networks (e.g., as described herein with respect to FIGS. 14 and 15). With reference to FIG. 10, at block 1004, an input image that depicts a section of a tissue and includes a plurality of artifact regions is accessed. At least one of the plurality of artifact regions depicts an anomaly that is not a structure of the tissue. At block 1008, a segmentation image is generated by processing the input image using a generator network. The segmentation image indicates, for each of the plurality of artifact regions of the input image, a boundary of the artifact region. The generator network has been trained using a training data set that includes a plurality of pairs of images, in which each pair includes a first image of a section of a tissue, the first image including at least one artifact region, and a second image that indicates, for each of the at least one artifact region of the first image, a boundary of the artifact region. In some embodiments, process 1000 also includes producing an annotated image that includes the segmentation image overlaid on the input image and/or estimating a quality of the input image, based on a total area of the plurality of artifact regions.

In some embodiments of process 1000, the anomaly is a focus blur, a fold in the section of the tissue, or a deposit of pigment in the section of the tissue.

In some embodiments of process 1000, the segmentation image comprises a binary segmentation mask.

In some embodiments of process 1000, the generator network is implemented as a fully convolutional network, as a U-Net, and/or as an encoder-decoder network.

In some embodiments of process 1000, the input image includes a second plurality of artifact regions, and the process also includes generating a second segmentation image by processing the input image using a second generator network, the second generator network having been trained using a second training data set that includes a second plurality of pairs of images. At least one of the second plurality of artifact regions depicts a biological structure of the tissue, and the second segmentation image indicates, for each of the second plurality of artifact regions of the input image, a boundary of the artifact region.

One or more methods according to the present disclosure may be implemented to relieve pathologists from the burden of manual delineation of at least some types of artifacts in the whole slide images, letting them focus on the viable tumor. For example, the task of manually delineating the artifacts may be simplified to performing a QC review on the results of an automatic artifact detection process as described herein. Such a process may be enabling for fully automated DP algorithms, as previous DP algorithms may not be capable of delineating the tumor area and exclusion regions on their own, thus creating a manual, tedious, and laborious preprocessing step for pathologists.

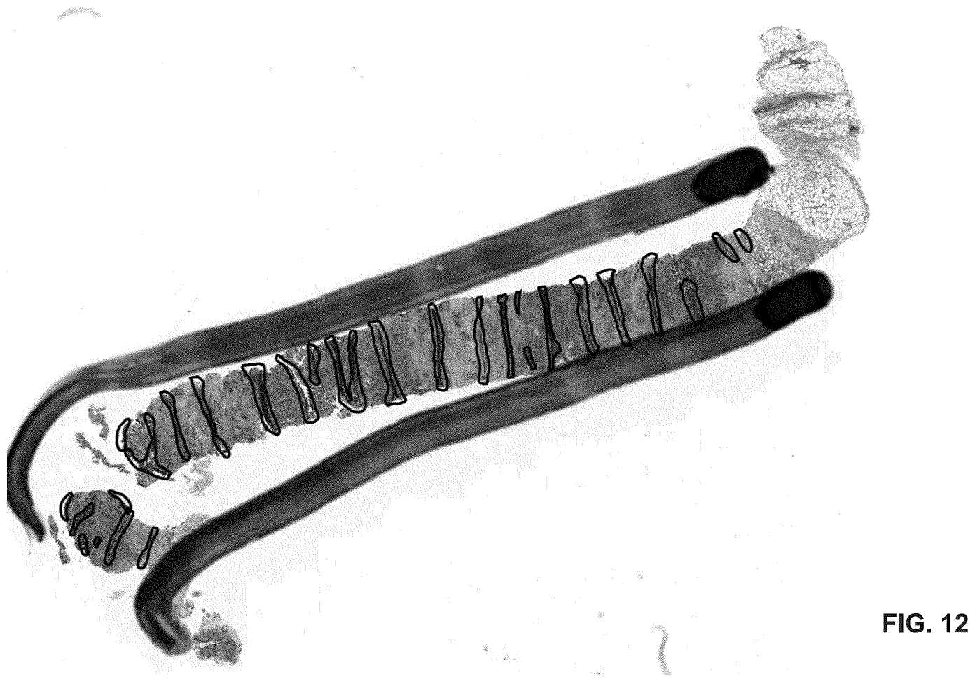

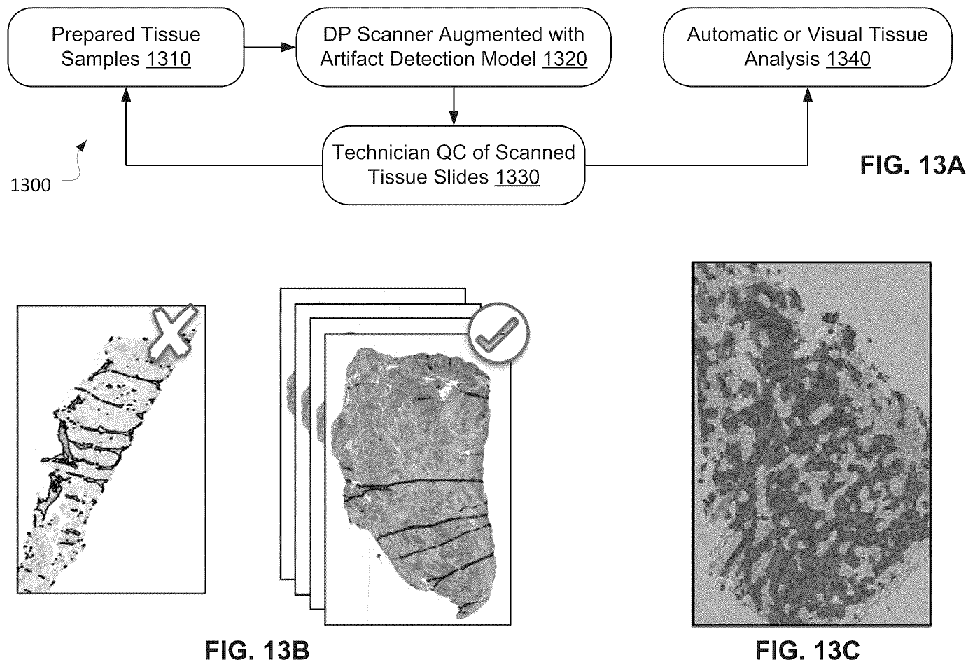

FIG. 11 shows an example of automated segmentation of tissue folds as produced by an implementation of process 1000. For comparison, FIG. 12 shows an example of a delineation of tissue folds as performed by human pathologists for the same slide image. It may be seen that the segmentation as produced by the algorithm is much more detailed and includes many smaller folds that the pathologists did not annotate at all. Moreover, the algorithm segmentation tracks the tissue folds very closely (thus minimizing the amount of area that would be incorrectly excluded), while the pathologists' tissue fold delineations include the neighboring region of each fold as well.

Since the manual work of delineating exclusion regions is very tedious and could take up to more than thirty minutes to finish one whole slide image, depending on the amount of exclusions that may be present, a pathologist may become less accurate or may miss small exclusion regions as the time spent on a slide begins to accumulate. Additionally, the inter- and intra-observer variability is another challenge that may affect the results of a subsequent analysis as well. In contrast, algorithms are highly reproducible, and their delineations are closer to the actual boundary of exclusion regions. Better boundary definition protects against spurious results from incomplete exclusion of unwanted regions, while preserving more of the desired region for analysis. The increased uniformity that may be provided by an automated solution as described herein may also be highly desirable for delineations that may be inherently subjective, such as delineation of a focus blur region.

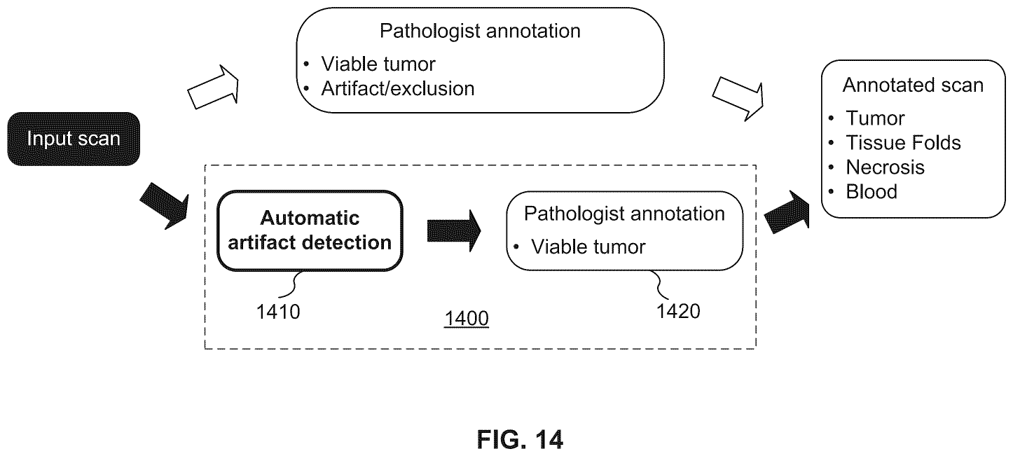

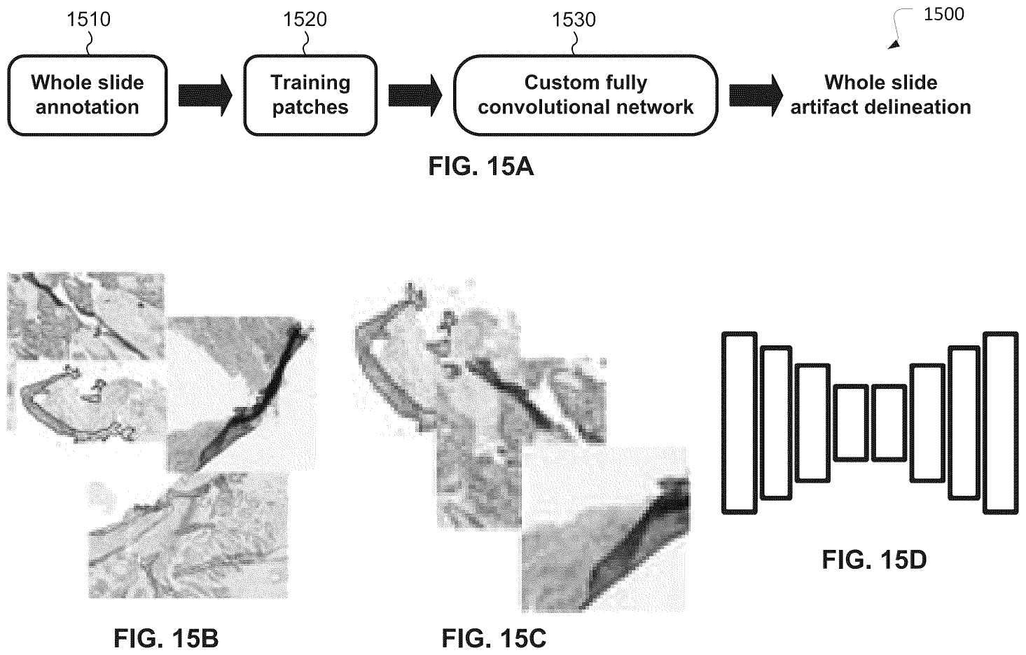

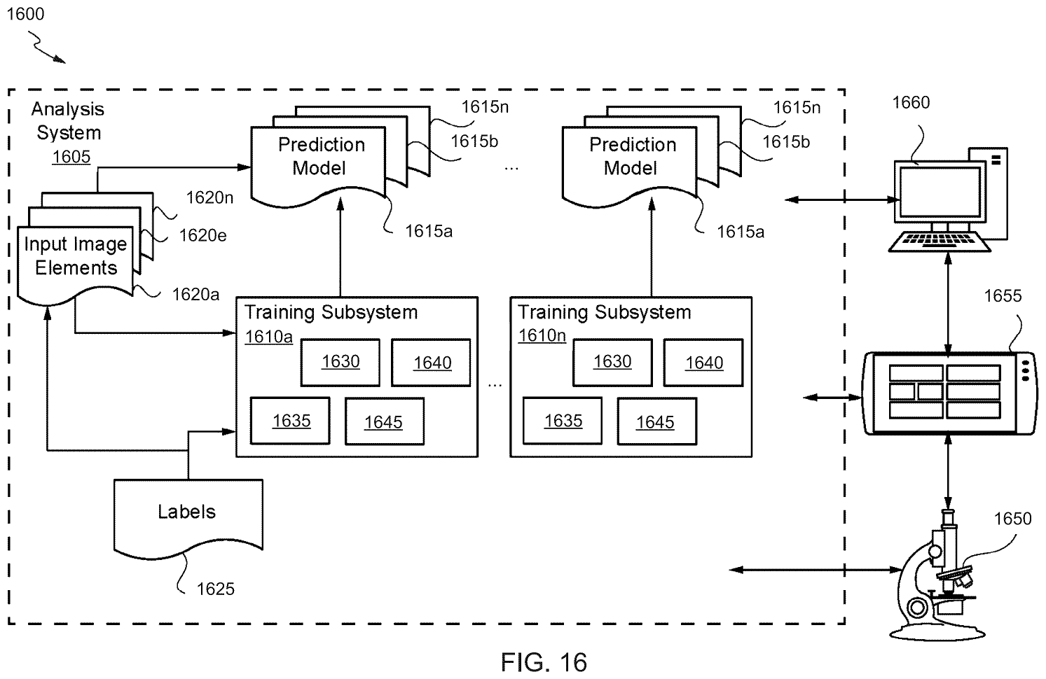

It may be desired to implement process 1000 to provide a scalable image quality control (QC) algorithm. FIG. 13A shows an example of such an application 1300 of process 1000 according to some embodiments. At block 1310, prepared tissue samples (e.g., prepared slides) are provided. At block 1320, the prepared samples are digitized (scanned) to obtain slide images (e.g., WSIs), using a DP scanner that is equipped with an artifact detection model. For example, the DP scanner may be configured to perform an embodiment of process 1300 as described herein. At block 1330, a technician performs a QC review of the annotated slide images (e.g., as shown in FIG. 13B). Slide images that pass the QC review are forwarded for automated or visual tissue analysis (e.g., as shown in FIG. 13C) at block 1340. Slide images that fail the QC review are rejected, and the process may return to block 1310 to re-scan the corresponding prepared sample (e.g., to correct a focus blur or stitching artifact) or for other action. For example, the sample may be cleaned and/or the slide may otherwise be re-prepared if possible (e.g., to correct artifacts due to unwanted matter, etc.), or the slide may be replaced using a new section of the sample. In another example of application 1300, block 1320 may be enhanced or replaced by a quality score that is calculated based on the segmentation image produced by process 1000. For example, process 1000 may be configured to calculate a quality score based on a total amount of the image area that is consumed by the artifact regions (alternatively, a total amount of the foreground area of the image that is consumed by the artifact regions, where the foreground area is the area occupied by the tissue section). In such case, the process may be configured to indicate a failing quality score if the area exceeds a threshold value.

It may be desired to implement process 1000 to provide automated exclusion of artifact regions (e.g., to reduce a pathologist workload). FIG. 14 shows an example of such an application 1400 of process 1000 according to some embodiments. In a previous workflow (top row), pathologist annotation of an input scan image (e.g., as shown in the example of FIG. 2B) includes annotation of one or more viable tumor regions (e.g., target regions) for analysis, and annotation of artifact regions and possibly other non-target regions for exclusion from the analysis (e.g., as shown in the example of FIG. 9). At block 1410, automatic artifact detection is performed (e.g., according to an implementation of process 1000 as described herein) to provide automated annotation of exclusion regions. The automatic artifact detection may be performed, for example, using a neural network architecture as depicted in FIG. 15D. At block 1420, the task of pathologist annotation of the input scan image includes annotation of viable tumor region(s) and is simplified by reducing the types of exclusion regions to be manually annotated or by removing the task of annotating exclusion regions.

FIG. 15A shows an example of a training workflow 1500 according to some embodiments. At block 1510, WSIs are manually annotated to indicate boundaries of artifact regions (e.g., as shown in FIG. 15B). At block 1520, the original (unannotated) and annotated WSIs are divided into training patches (e.g., of size 64x64, 128x128, or 256x256 pixels) (as shown, for example, in FIG. 15B), and one or more training sets of matched pairs of images are prepared. Each matched pair includes an unannotated version of a corresponding image patch and an annotated version of the same image patch. At block 1530, the matched pairs of the one or more training sets are used to train a custom fully convolutional network (e.g., as depicted in FIG. 15D) to perform image segmentation, and the trained network is used to perform automated delineation of artifact regions in a digital pathology slide (e.g., WSI).

It may be desired to train the network to be scanner-agnostic, tissue-agnostic, and/or stain-agnostic, as image artifacts (e.g., anomalies) may happen regardless of the brand of scanner, the tissue indication, or the staining used. In order to ensure that the detection algorithm is robust to different scanners, tissue types, staining types, and preparation protocols, it may be desired to train the deep-learning model using images from different bright field microscopy (e.g., H&E, IHC PD-L1 and epidermal growth factor receptor (EGFR)), different tissues (e.g., lung and colon), and different scanners (e.g., VENTANA DP200 and VENTANA Aperio).

As described above, it may be desired to train the network to map input images to corresponding segmentation images using supervised learning (e.g., based on example original-annotated image patch pairs). Supervised learning may include penalizing the model for making mistakes in terms of mispredicting or otherwise mismatching the generated output segmentation mask with the available “ground truth” mask (e.g., the manually annotated image patch).

In one example, the network is trained on different anomalies (e.g., tissue-fold artifacts and focus blur artifacts) at the same time (e.g., as a single “exclude” class). In such a context of supervised learning, however, it may be desired to treat some different types of artifacts separately (e.g., as different classes). For example, it may be desired to separate training with respect to artifacts that depict anomalies that are not structures of the tissue from training with respect to artifacts that depict biological structures of the tissue, both in terms of the output of the network as well as providing separate ground truths for each of the desired classes of artifact. In one example, a prediction model 1415 is implemented to support segmentation of both anomalies and unwanted biological structures by modifying the last layer of the network to support multi-class output.

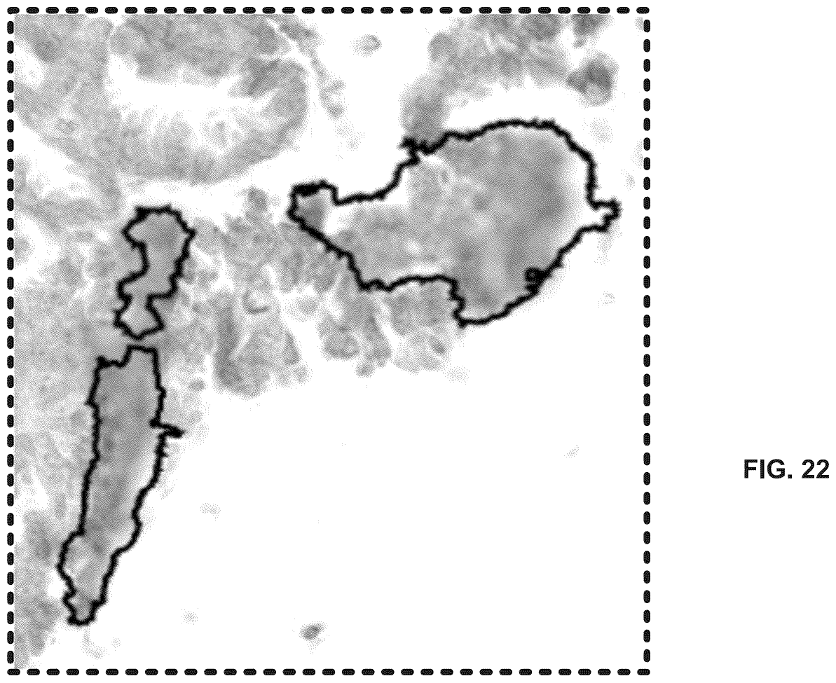

FIG. 16 shows a block diagram that illustrates an example computing environment 1600 (i.e., a data processing system) for segmenting an input image according to various embodiments. The computing environment 1600 can include an analysis system 1605 to train and execute prediction models, e.g., two-dimensional CNN models. More specifically, the analysis system 1605 can include training subsystems 1610a-n (‘a’ and ‘n’ represents any natural number) that build and train their respective prediction models 1615a-n (which may be referred to herein individually as a prediction model 1615 or collectively as the prediction models 1615) to be used by other components of the computing environment 1600. A prediction model 1615 can be a machine-learning (“ML”) model, such as a deep convolutional neural network (CNN), e.g. an inception neural network, a residual neural network (“Resnet”), or a recurrent neural network, e.g., long short-term memory (“LSTM”) models or gated recurrent units (“GRUs”) models. A prediction model 1615 can also be any other suitable ML model trained to segment non-target regions (e.g., artifact regions), segment target regions, or provide image analysis of target regions, such as a two-dimensional CNN (“2DCNN”), a Mask R-CNN, Feature Pyramid Network (FPN), a dynamic time warping (“DTW”) technique, a hidden Markov model (“HMM”), etc., or combinations of one or more of such techniques—e.g., CNN-HMM or MCNN (Multi-Scale Convolutional Neural Network). The computing environment 1600 may employ the same type of prediction model or different types of prediction models trained to segment non-target regions, segment target regions, or provide image analysis of target regions. For example, computing environment 1600 can include a first prediction model (e.g., a U-Net) for segmenting non-target regions (e.g., artifact regions). The computing environment 1600 can also include a second prediction model (e.g., a 2DCNN) for segmenting target regions (e.g., regions of tumor cells). The computing environment 1600 can also include a third model (e.g., a CNN) for image analysis of target regions. The computing environment 1600 can also include a fourth model (e.g., a HMM) for diagnosis of disease for treatment or a prognosis for a subject such as a patient. Still other types of prediction models may be implemented in other examples according to this disclosure.

In various embodiments, each prediction model 1615a-n corresponding to the training subsystems 1610a-n is separately trained based on one or more sets of input image elements 1620a-n. In some embodiments, each of the input image elements 1620a-n include image data from one or more scanned slides. Each of the input image elements 1620a-n may correspond to image data from a single specimen and/or a single day on which the underlying image data corresponding to the image was collected. The image data may include an image, as well as any information related to an imaging platform on which the image was generated. For instance, a tissue section may need to be stained by means of application of a staining assay containing one or more different biomarkers associated with chromogenic stains for brightfield imaging or fluorophores for fluorescence imaging. Staining assays can use chromogenic stains for brightfield imaging, organic fluorophores, quantum dots, or organic fluorophores together with quantum dots for fluorescence imaging, or any other combination of stains, biomarkers, and viewing or imaging devices. Moreover, a typical tissue section is processed in an automated staining/assay platform that applies a staining assay to the tissue section, resulting in a stained sample. There are a variety of commercial products on the market suitable for use as the staining/assay platform, one example being the VENTANA SYMPHONY product of the assignee Ventana Medical Systems, Inc.

Stained tissue sections may be supplied to an imaging system, for example on a microscope or a whole-slide scanner having a microscope and/or imaging components, one example being the VENTANA iScan Coreo product of the assignee Ventana Medical Systems, Inc. Multiplex tissue slides may be scanned on an equivalent multiplexed slide scanner system. Additional information provided by the imaging system may include any information related to the staining platform, including a concentration of chemicals used in staining, a reaction times for chemicals applied to the tissue in staining, and/or pre-analytic conditions of the tissue, such as a tissue age, a fixation method, a duration, how the section was embedded, cut, etc.

The input image elements 1620a-n may include one or more training input image elements 1620a-d, validation input image elements 1620e-g, and unlabeled input image elements 1620h-n. It should be appreciated that input image elements 1620a-n corresponding to the training, validation and unlabeled groups need not be accessed at a same time. For example, an initial set of training and validation input image elements 1620a-n may first be accessed and used to train a prediction model 1615, and unlabeled input image elements may be subsequently accessed or received (e.g., at a single or multiple subsequent times) and used to by a trained prediction model 1615 to provide desired output (e.g., segmentation of non-target regions). In some instances, the prediction models 1615a-n are trained using supervised training, and each of the training input image elements 1620a-d and optionally the validation input image elements 1620e-g are associated with one or more labels 1625 that identify a “correct” interpretation of non-target regions, target regions, and identification of various biological material and structures within training input image elements 1620a-d and the validation input image elements 1620e-g. Labels may alternatively or additionally be used to classify a corresponding training input image elements 1620a-d and the validation input image elements 1620e-g, or pixel therein, with regards to a presence and/or interpretation of a stain associated with a normal or abnormal biological structure (e.g., a tumor cell). In certain instances, labels may alternatively or additionally be used to classify a corresponding training input image elements 1620a-d and the validation input image elements 1620e-g at a time point corresponding to when the underlying image was/were taken or a subsequent time point (e.g., that is a predefined duration following a time when the image(s) was/were taken).

In some embodiments, the training subsystems 1610a-n include a feature extractor 1630, a parameter data store 1635, a classifier 1640, and a trainer 1645, which are collectively used to train the prediction models 1615 based on training data (e.g., the training input image elements 1620a-d) and optimizing the parameters of the prediction models 1615 during supervised or unsupervised training. In some instances, the training process includes iterative operations to find a set of parameters for the prediction model 1615 that minimizes a loss function for the prediction models 1615. Each iteration can involve finding a set of parameters for the prediction model 1615 so that the value of the loss function using the set of parameters is smaller than the value of the loss function using another set of parameters in a previous iteration. The loss function can be constructed to measure the difference between the outputs predicted using the prediction models 1615 and the labels 1625 contained in the training data. Once the set of parameters are identified, the prediction model 1615 has been trained and can be utilized for segmentation and/or prediction as designed.

In some embodiments, the training subsystem 1610a-n accesses training data from the training input image elements 1620a-d at the input layers. The feature extractor 1630 may pre-process the training data to extract relevant features (e.g., edges) detected at particular parts of the training input image elements 1620a-d. The classifier 1640 can receive the extracted features and transform the features, in accordance with weights associated with a set of hidden layers in one or more prediction models 1615, into one or more output metrics that segment non-target or target regions, provide image analysis, provide a diagnosis of disease for treatment or a prognosis for a subject such as a patient, or a combination thereof. The trainer 1645 may use training data corresponding to the training input image elements 1620a-d to train the feature extractor 1630 and/or the classifier 1640 by facilitating learning of one or more parameters. For example, the trainer 1645 can use a backpropagation technique to facilitate learning of weights associated with a set of hidden layers of the prediction model 1615 used by the classifier 1640. The backpropagation may use, for example, a stochastic gradient descend (SGD) algorithm to cumulatively update the parameters of the hidden layers. Learned parameters may include, for instance, weights, biases, and/or other hidden layer-related parameters, which can be stored in the parameter data store 1635.

Individually or an ensemble of trained prediction models can be deployed to process unlabeled input image elements 1620h-n to segment non-target or target regions, provide image analysis, provide a diagnosis of disease for treatment or a prognosis for a subject such as a patient, or a combination thereof. More specifically, a trained version of the feature extractor 1630 may generate a feature representation of an unlabeled input image element, which can then be processed by a trained version of the classifier 1640. In some embodiments, image features can be extracted from the unlabeled input image elements 1620h-n based on one or more convolutional blocks, convolutional layers, residual blocks, or pyramidal layers that leverage dilation of the prediction models 1615 in the training subsystems 1610a-n. The features can be organized in a feature representation, such as a feature vector of the image. The prediction models 1615 can be trained to learn the feature types based on classification and subsequent adjustment of parameters in the hidden layers, including a fully connected layer of the prediction models 1615.

In some embodiments, the image features extracted by the convolutional blocks, convolutional layers, residual blocks, or pyramidal layers include feature maps that are matrices of values that represent one or more portions of the specimen slide at which one or more image processing operations have been performed (e.g., edge detection, sharpen image resolution). These feature maps may be flattened for processing by a fully connected layer of the prediction models 1615, which outputs a non-target region mask, target region mask, or one or more metrics corresponding to a present or future prediction pertaining to a specimen slide. For example, an input image element can be fed to an input layer of a prediction model 1615. The input layer can include nodes that correspond with specific pixels. A first hidden layer can include a set of hidden nodes, each of which is connected to multiple input-layer nodes. Nodes in subsequent hidden layers can similarly be configured to receive information corresponding to multiple pixels. Thus, hidden layers can be configured to learn to detect features extending across multiple pixels. Each of one or more hidden layers can include a convolutional block, convolutional layer, residual block, or pyramidal layer. The prediction model 1615 can further include one or more fully connected layers (e.g., a softmax layer).

At least part of the training input image elements 1620a-d, the validation input image elements 1620e-g and/or the unlabeled input image elements 1620h-n may include or may have been derived from data obtained directly or indirectly from a source that may be but need not be an element of the analysis system 1605. In some embodiments, the computing environment 1600 comprises an imaging device 1650 that images a sample to obtain the image data, such as a multi-channel image (e.g., a multi-channel fluorescent or brightfield image) with several (such as between ten to sixteen for example) channels. The image device 1650 may include, without limitation, a camera (e.g., an analog camera, a digital camera, etc.), optics (e.g., one or more lenses, sensor focus lens groups, microscope objectives, etc.), imaging sensors (e.g., a charge-coupled device (CCD), a complimentary metal-oxide semiconductor (CMOS) image sensor, or the like), photographic film, or the like. In digital embodiments, the image capture device can include a plurality of lenses that cooperate to provide on-the-fly focusing. An image sensor, for example, a CCD sensor can capture a digital image of the specimen. In some embodiments, the imaging device 1650 is a brightfield imaging system, a multispectral imaging (MSI) system or a fluorescent microscopy system. The imaging device 1650 may utilize nonvisible electromagnetic radiation (UV light, for example) or other imaging techniques to capture the image. For example, the imaging device 1650 may comprise a microscope and a camera arranged to capture images magnified by the microscope. The image data received by the image analysis system 1605 may be identical to and/or derived from raw image data captured by the imaging device 1650.

In some instances, labels 1625 associated with the training input image elements 1620a-d and/or validation input image elements 1620e-g may have been received or may be derived from data received from one or more provider systems 1655, each of which may be associated with (for example) a physician, nurse, hospital, pharmacist, etc. associated with a particular subject. The received data may include (for example) one or more medical records corresponding to the particular subject. The medical records may indicate (for example) a professional's diagnosis or characterization that indicates, with respect to a time period corresponding to a time at which one or more input image elements associated with the subject were collected or a subsequent defined time period, whether the subject had a tumor and/or a stage of progression of the subject's tumor (e.g., along a standard scale and/or by identifying a metric, such total metabolic tumor volume (TMTV)). The received data may further include the pixels of the locations of tumors or tumor cells within the one or more input image elements associated with the subject. Thus, the medical records may include or may be used to identify, with respect to each training/validation input image element 1620a-g, one or more labels. The medical records may further indicate each of one or more treatments (e.g., medications) that the subject had been taking and time periods during which the subject was receiving the treatment(s). In some instances, images or scans that are input to one or more training subsystems are received from the provider system 1655. For example, the provider system 1655 may receive images from the imaging device 1650 and may then transmit the images or scans (e.g., along with a subject identifier and one or more labels) to the analysis system 1605.

In some embodiments, data received at or collected at one or more of the imaging devices 1650 may be aggregated with data received at or collected at one or more of the provider systems 1655. For example, the analysis system 1605 may identify corresponding or identical identifiers of a subject and/or time period so as to associate image data received from the imaging device 1650 with label data received from the provider system 1655. The analysis system 1605 may further use metadata or automated image analysis to process data to determine to which training subsystem particular data components are to be fed. For example, image data received from the imaging device 1650 may correspond to the whole slide or multiple regions of the slide or tissue. Metadata, automated alignments and/or image processing may indicate, for each image, to which region of the slide or tissue the image corresponds. For example, automated alignments and/or image processing may include detecting whether an image has image properties corresponding to a slide substrate or a biological structure and/or shape that is associated with a particular cell such as a white blood cell. Label-related data received from the provider system 1655 may be slide-specific, region-specific or subject-specific. When label-related data is slide-specific or region specific, metadata or automated analysis (e.g., using natural language processing or text analysis) can be used to identify to which region particular label-related data corresponds. When label-related data is subject-specific, identical label data (for a given subject) may be fed to each training subsystem 1610a-n during training.

In some embodiments, the computing environment 1600 can further include a user device 1660, which can be associated with a user that is requesting and/or coordinating performance of one or more iterations (e.g., with each iteration corresponding to one run of the model and/or one production of the model's output(s)) of the analysis system 1605. The user may correspond to a physician, investigator (e.g., associated with a clinical trial), subject, medical professional, etc. Thus, it will be appreciated that, in some instances, the provider system 1655 may include and/or serve as the user device 1660. Each iteration may be associated with a particular subject (e.g., person), who may (but need not) be different than the user. A request for the iteration may include and/or be accompanied with information about the particular subject (e.g., a name or other identifier of the subject, such as a de-identified patient identifier). A request for the iteration may include an identifier of one or more other systems from which to collect data, such as input image data that corresponds to the subject. In some instances, a communication from the user device 1660 includes an identifier of each of a set of particular subjects, in correspondence with a request to perform an iteration for each subject represented in the set.

Upon receiving the request, the analysis system 1605 can send a request (e.g., that includes an identifier of the subject) for unlabeled input image elements to the one or more corresponding imaging systems 1650 and/or provider systems 1655. The trained prediction model(s) 1615 can then process the unlabeled input image elements to segment non-target or target regions, provide image analysis, provide a diagnosis of disease for treatment or a prognosis for a subject such as a patient, or a combination thereof. A result for each identified subject may include or may be based on the segmenting and/or one or more output metrics from trained prediction model(s) 1615 deployed by the training subsystems 1610a-n. For example, the segmenting and/or one or more output metrics can include or may be based on output generated by the fully connected layer of one or more CNNs. In some instances, such outputs may be further processed using (for example) a softmax function. Further, the outputs and/or further processed outputs may then be aggregated using an aggregation technique (e.g., random forest aggregation) to generate one or more subject-specific metrics. One or more results (e.g., that include plane-specific outputs and/or one or more subject-specific outputs and/or processed versions thereof) may be transmitted to and/or availed to the user device 1660. In some instances, some or all of the communications between the analysis system 1605 and the user device 1660 occurs via a website. It will be appreciated that the CNN system 1605 may gate access to results, data and/or processing resources based on an authorization analysis.

While not explicitly shown, it will be appreciated that the computing environment 1600 may further include a developer device associated with a developer. Communications from a developer device to components of the computing environment 1600 may indicate what types of input images are to be used for each prediction model 1615 in the analysis system 1605, a number and type of models to be used, hyperparameters of each model (for example, learning rate and number of hidden layers), how data requests are to be formatted, which training data is to be used (e.g., and how to gain access to the training data) and which validation technique is to be used, and/or how the controller processes are to be configured.

As noted above, a prediction model 1615 may be implemented using a U-Net architecture, which includes an encoder having layers that progressively downsample the input to a bottleneck layer, and a decoder having layers that progressively upsample the bottleneck output to produce the output. A U-Net also includes skip connections between encoder and decoder layers having equally sized feature maps; these connections concatenate the channels of the feature map of the encoder layer with those of the feature map of the corresponding decoder layer. In a particular example, the prediction model 1615 is updated via a cross-entropy loss measured between the generated image and the expected output image (e.g., the “predicted image” and the “ground truth,” respectively). Other examples of a loss function that may be used to update the prediction model 1615 include, e.g., an L1 loss or an L2 loss.

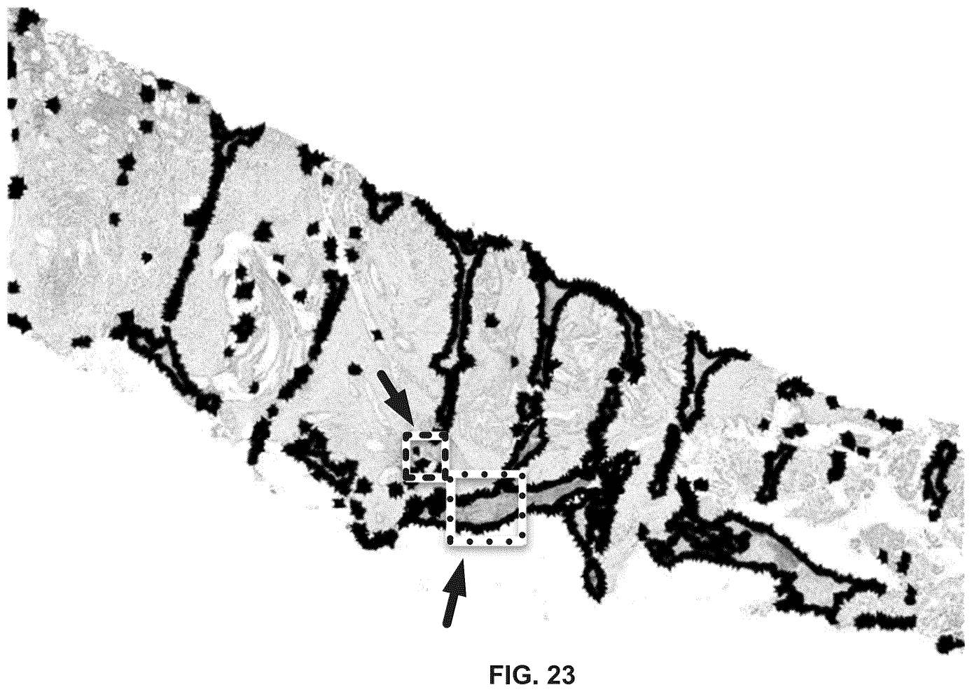

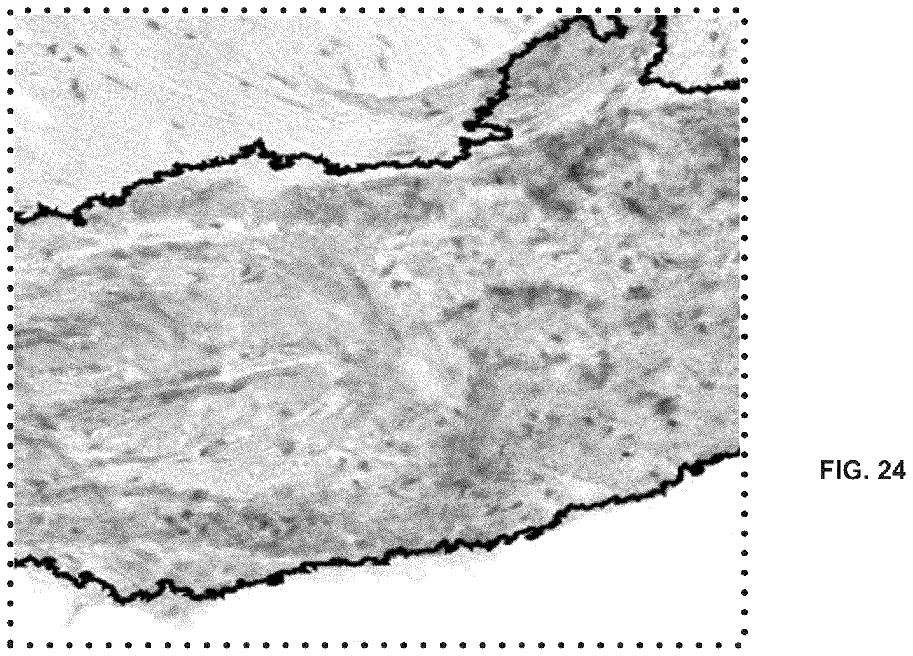

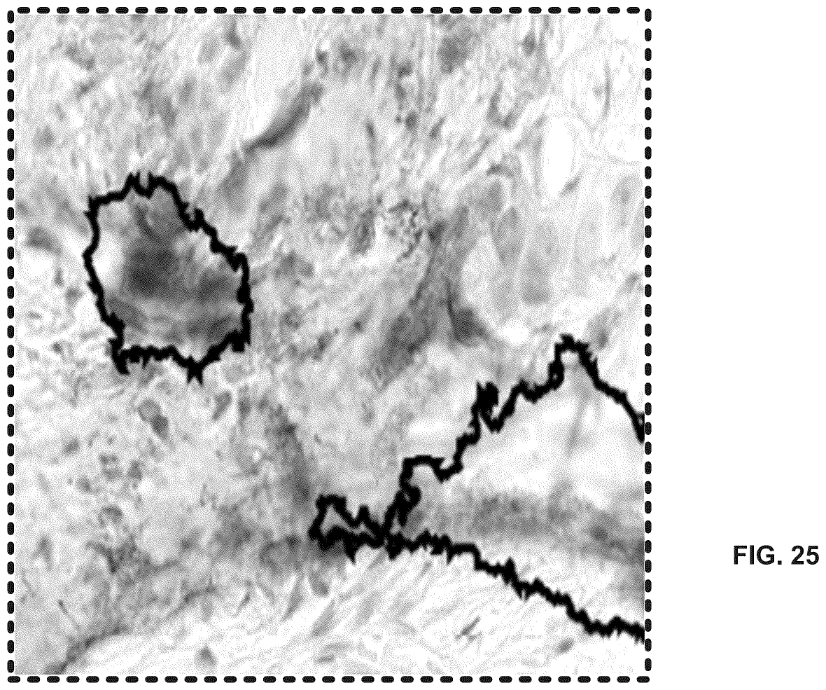

FIGS. 17-25 illustrate example results of an implementation of process 1000, in which FIGS. 18, 21, and 24 are enlarged versions of the areas indicated by the dotted outlines in FIGS. 17, 20, and 23, respectively, and FIGS. 19, 22, and 25 are enlarged versions of the areas indicated by the dashed outlines in FIGS. 17, 20, and 23, respectively. In this example, the model was trained on tissue-fold artifacts and focus blur artifacts, and the trained model was tested on a variety of whole slide images coming from different scanners, different stains, and different tissue types. As described herein, the method may be extended to other common tissue-based or image-based artifacts as well.

It can be seen that the algorithm identifies many small regions with out-of-focus issues (see especially, e.g., FIGS. 22 and 25) which would be almost impossible for a human pathologist to delineate in a similar manner without spending hours on the same slide. Additionally, as discussed above with reference to FIGS. 11 and 12, it may be seen that the algorithm draws the segmentation boundaries very close to the actual boundary of the artifacts. Such precision is not feasible for a human pathologist without going to high magnifications of 40× (thus greatly increasing the size of the image area to be reviewed) and spending more time following the boundaries of the artifacts. Such a method may provide an automated solution for segmentation of artifacts in DP images that is more scalable, more robust, and cheaper as compared to pathologist manual annotations.

V. Additional Considerations

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The description provides preferred exemplary embodiments only, and is not intended to limit the scope, applicability or configuration of the disclosure. Rather, the description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed is:

1. A method of image segmentation, the method comprising:
accessing an input image that depicts a section of a tissue and includes a plurality of artifact regions; and
generating a segmentation image by processing the input image using a generator network, the generator network having been trained using a training data set that includes a plurality of pairs of images,
wherein the segmentation image indicates, for each of the plurality of artifact regions of the input image, a boundary of the artifact region, and
wherein at least one of the plurality of artifact regions depicts an anomaly that is not a structure of the tissue, and
wherein, for each pair of images of the plurality of pairs of images, the pair includes:
a first image of a section of a tissue, the first image including at least one artifact region, and
a second image that indicates, for each of the at least one artifact region of the first image, a boundary of the artifact region.

2. The method of claim 1, wherein the anomaly is a focus blur.

3. The method of claim 1, wherein the anomaly is a fold in the section of the tissue.

4. The method of claim 1, wherein the anomaly is a deposit of pigment in the section of the tissue.

5. The method of claim 1, wherein the segmentation image comprises a binary segmentation mask.

6. The method of claim 1, wherein the method further comprises producing an annotated image that includes the segmentation image overlaid on the input image.

7. The method of claim 1, wherein the method further comprises estimating a quality of the input image, based on a total area of the plurality of artifact regions.

8. The method of claim 1, wherein the input image includes a second plurality of artifact regions, and wherein the method further comprises:
generating a second segmentation image by processing the input image using a second generator network, the second generator network having been trained using a second training data set that includes a second plurality of pairs of images,
wherein the second segmentation image indicates, for each of the second plurality of artifact regions of the input image, a boundary of the artifact region, and
wherein at least one of the second plurality of artifact regions depicts a biological structure of the tissue.

9. The method of claim 1, wherein the generator network is implemented as a fully convolutional network.

10. The method of claim 1, wherein the generator network is implemented as a U-Net.

11. The method of claim 1, wherein the generator network is implemented as an encoder-decoder network.

12. The method of claim 1, wherein the generator network is updated via a cross-entropy loss measured between an image by the generator network and an expected output image.

13. The method of claim 1, further comprising: determining, by a user, a diagnosis of a subject based on the segmentation image.

14. The method of claim 13, further comprising administering, by the user, a treatment with a compound based on (i) the segmentation image, and/or (ii) the diagnosis of the subject.

15. A system comprising:
one or more data processors; and
a non-transitory computer readable storage medium containing instructions which, when executed on the one or more data processors, cause the one or more data processors to perform a method comprising:
accessing an input image that depicts a section of a tissue and includes a plurality of artifact regions; and
generating a segmentation image by processing the input image using a generator network, the generator network having been trained using a training data set that includes a plurality of pairs of images,
wherein the segmentation image indicates, for each of the plurality of artifact regions of the input image, a boundary of the artifact region, and
wherein at least one of the plurality of artifact regions depicts an anomaly that is not a structure of the tissue, and
wherein, for each pair of images of the plurality of pairs of images, the pair includes:
a first image of a section of a tissue, the first image including at least one artifact region, and
a second image that indicates, for each of the at least one artifact region of the first image, a boundary of the artifact region.

16. The system of claim 15, wherein the anomaly is a focus blur, a fold in the section of the tissue, or a deposit of pigment in the section of the tissue.

17. The system of claim 15, wherein the generator network is implemented as a fully convolutional network, a U-Net, or an encoder-decoder network.

18. A computer-program product tangibly embodied in a non-transitory machine-readable storage medium, including instructions configured to cause one or more data processors to perform a method comprising:
accessing an input image that depicts a section of a tissue and includes a plurality of artifact regions; and
generating a segmentation image by processing the input image using a generator network, the generator network having been trained using a training data set that includes a plurality of pairs of images,
wherein the segmentation image indicates, for each of the plurality of artifact regions of the input image, a boundary of the artifact region, and
wherein at least one of the plurality of artifact regions depicts an anomaly that is not a structure of the tissue, and
wherein, for each pair of images of the plurality of pairs of images, the pair includes:
a first image of a section of a tissue, the first image including at least one artifact region, and
a second image that indicates, for each of the at least one artifact region of the first image, a boundary of the artifact region.

19. The computer-program product of claim 18, wherein the anomaly is a focus blur, a fold in the section of the tissue, or a deposit of pigment in the section of the tissue.

20. The computer-program product of claim 18, wherein the generator network is implemented as a fully convolutional network, a U-Net, or an encoder-decoder network.
